(19) United States
(12) Patent Application Publication
(10) Pub. No.: US 2002/0128623 A1
(43) Pub. Date: Sep. 12, 2002

(54) Disposal human waste containment contrivance

(76) Inventor: Thomas Kehoe, Miami, FL (US)

Correspondence Address: Richard S. Ross, Esq., Atrium Center, 4801 South University Drive, Suite 3070, Ft. Lauderdale, FL, 33328, US

(21) Appl. No.: 09/802,554

(22) Filed: Mar. 12, 2001

(51) Int. Cl. A61F 13/15

(52) U.S. Cl. 604/385.19; 604/385.09; 604/327; 604/348

(57) ABSTRACT

A disposable, undergarment-like panty/brief for collection and retention of fecal matter. The disposable, undergarment-like panty/brief of this invention comprises a waist band equipped with a draw-string, a back portion or panel, a front portion or panel, a seat/crotch/sump. Each of the back portion or panel and front portion or panel is attached at its corresponding top margins to the waist band and further cooperate with each other at each of its corresponding lateral margins, so as to generally define leg openings in the undergarment-like brief or panty. The front and back portions or panels also cooperate with each other at each of its corresponding bottom margins to define an extended crotch or seat. The extended sump is configured to remain remote from the buttocks of the wearer and thereby provides a container for the collection of human waste. Thus, in application or use of the disposal undergarment-like brief or panty, the panty/brief is put on by the user, the user causes the excretion of waste matter into the panty/brief and thereafter removes the panty/brief. The waste matter can be sealed within the panty/brief by twisting the panty/brief and securing the twist with the drawstring of the waist band.

### BACKGROUND OF THE INVENTION

1. Field of the Invention

This invention relates to an article of manufacture. More specifically, this invention relates to a human waste containment contrivance in the form of a disposable, undergarment-like panty/brief for collection and retention of fecal matter.

2. Background of Invention

The adaptation of sanitary waste disposal devices to recreation or emergency settings has been previously disclosed in the technical and patent literature. Generally, such waste disposal involves the use of a temporary commode and a disposal waste container associated with the commode.

The following patents are representative of such temporary commodes and waste disposal systems employing a disposable liner/bag associated with such prior art devices. Each of these patents is discussed in chronological order, based upon its date of issue, and, thus, no added significance is to be attached to its order of discussion.

U.S. Pat. No. 3,484, 875 (to Eisenberg, issued Dec. 23, 1969) describes a collapsible portable commode comprising a collapsible seat, a re-enforceable plastic member (tubular support for the seat) and a removable plastic bag provided with a drawstring. In practice, the re-enforceable plastic member is first deployed, the plastic bag draped over the tubular lip of the support member and the seat thereafter installed. The pressure fit of the seat on the tubular support member retains the removable plastic bag in place when the commode is in use, and thereafter is replaced with a clean bag. The drawstring on the used bag is use to seal the waste in the bag for later disposal.

U.S. Pat. No. 4,996,727 (to Wyatt, issued Mar. 5, 1991) describes a disposable waste bag which is usable independently or with portable commodes, hospital commodes, conventional bedpans and the like. The Wyatt disposable waste bag comprises a composite having a first circularly shaped sheet of a liquid-impermeable material. A second circularly shaped sheet of a liquid-tight material is also provided, with the second sheet being secured to the first sheet along a portion of their respective perimeters to define a waste containing enclosure having an open end and a closed end. The resultant enclosure is further laminated to a sheet of semi-rigid material. The semi-rigid sheet material has an opening in the center thereof which conforms to the size of the open end of the waste containing enclosure, with the open end of the enclosure being secured to the perimeter of the opening in the semi-rigid material. The semi-rigid sheet of the resultant composite is further provided with means for folding the semi-rigid sheet in half to effect closure and sealing of the waste containing enclosure.

U.S. Pat. No. 5,095,556 (to Franey, issued Mar. 17, 1992) describes a foldable, portable personal commode having a toilet seat which is pivotally mounted to an extension or brace within a carrying/storage case. According to Franey, a suitcase-like carrying case is provided for storage, deployment and support of a portable commode. In brief, upon opening the carrying case, the Franey device is deployed by an assembly of the various components contained within the carrying case. The assembled commode includes components of the carrying case as part of its integral supporting structure. The assembled commode includes a disposable waste containment bag that is to be releasably engaged to the underside of the supporting structure of the toilet seat. Accordingly, subsequent to use, the waste disposable bag is removed, sealed for safe disposal at a later time. The disposable bag is, thereafter, replaced.

U.S. Pat. No. 5,155,871 (to Sams, issued Oct. 20, 1992) describes a portable chamber-pot having a generally annular member for supporting a disposable container, the annular member defining a seating surface with an opening therein, and having inner and outer skirts extending generally normal to said seating surface. The liner of the Sams device has a pair of loops or handles which can be used to secure the liner to the chamber-pot by looping the handles around the side walls. The liner comprises a tube of plastics material which is flattened and provided with a side pleat along each of its opposite sides. Each of the sides of the line includes a pair of slots which form/define a pair of handles which are arranged to secure the liner to the side walls of the chamber-pot and, when removed from the chamber-pot, to carry the contents deposited therein securely for subsequent disposal. Alternatively, the handles may be knotted together to seal the contents inside the liner before disposal.

As is evident from the above discussion, each of the portable commodes described in the prior art uses, in one form or another, a disposable bag for collection and containment of the human waste. In each instance the disposable bag is designed to be releasably secured to the toilet seat, or the equivalent, and thereafter disengaged for disposal. In the limited instances where a waste disposal device has been disclosed for use independent of a portable commode, (e.g. U.S. Pat. No. 4,312,085), it does not provide comparable convenience or sanitation. Accordingly, there continues to exist a need to provide an improved sanitation device for containment and disposal of human waste, which is preferably suitable for use independent of a portable commode.

### OBJECTS OF THE INVENTION

It is the object of this invention to remedy the above as well as related deficiencies in the prior art.

More specifically, it is the principal object of the present invention to provide an undergarment-like contrivance that is suitable for collection and disposal of human waste, independent of other supporting means or commode-like devices.

It is another object of this invention to provide an undergarment-like contrivance, in the form of a disposable panty/brief, which, by design, performs its function and yet maintains the collected human waste remote from contact with the user of the contrivance.

It is yet another object of this invention to provide an undergarment-like contrivance, in the form of a disposable panty/brief, that can be used to both collect human waste and toilet tissue in essentially the same manner as a conventional toilet, yet perform such function independent of other supporting means or a commode-like devices.

### SUMMARY OF THE INVENTION

The above and related objects are achieved by providing a disposable, undergarment-like panty/brief for collection and retention of human waste. In the preferred embodiments of this invention, the disposable, undergarment-like panty/brief comprises a waist band equipped with a draw-string, a back portion or panel, a front portion or panel, and a seat/crotch/sump. Each of the back portion or panel and front portion or panel is attached at its corresponding top margins to the waist band and further cooperate with each other at each of its corresponding lateral margins, so as to generally define leg openings in the undergarment-like panty/brief The front and back portions or panels also cooperate with each other at each of its corresponding bottom margins to define an extended crotch, seat or sump. The extended crotch or seat is configured to remain remote from the buttocks of the wearer and thereby provides a container or sack for the collection of human waste. Accordingly, in application or use of the disposal undergarment-like panty/brief, the panty/brief is initially put on by the user at the time of contemplated use, the user causes the excretion of waste matter into the panty/brief and thereafter removes the brief or panty. The waste matter can be sealed within the panty/brief by twisting the panty/brief and then tying the resulting twist with the draw-string of the waist band.

In one of the preferred embodiments of this invention, a portion of the front panel, which defines each of the leg openings, is releasably attached to the waist band at the top margin of the front panel of the panty/brief, so as to permit the panty/brief to be put on and removed without removal of the user's trousers or pants.

In another of the preferred embodiments of this invention, the back panel of the panty/brief has an aperture of sufficient size to permit the user to wipe himself/herself with toilet tissue, while still wearing the brief or panty. The used toilet tissue is discarded within the panty/brief in the same manner as would normally occur in the use of a commode.

The panty/brief can be optionally be pre-packaged with toilet tissue, be provided with a deodorizer or other means to accommodate its disposal (e.g. Zip Lock bag) or include a high fluid capacity absorbent polymer/fabric to prevent possible loss of liquid waste from within the brief or panty.

### BRIEF DESCRIPTION OF THE DRAWINGS

FIG. 1 is a perspective view of the disposable, undergarment-like panty/brief of this invention.

FIG. 2 is a perspective view of the disposable, undergarment-like panty/brief of FIG. 1, wherein the front panel is releasably detached from the waist band to permit it to put on and removed by the user without complete removal of his/her trousers.

FIGS. 3A & 3B each provide a perspective view of alternative embodiments of the disposable, undergarment-like panty/brief of FIG. 1, wherein the front panel is releasably detached from the waist band to permit it to put on and removed by the user without complete removal of his/her trousers.

FIGS. 4A & 4B each provide a perspective view of the disposable, undergarment-like panty/brief of FIG. 1, wherein the panty/brief has been used (FIG. 4A) and sealed (FIG. 4B) prior to disposal.

FIGS. 5A, 5B & 5C provide a perspective view of the disposable, undergarment-like panty/brief of FIG. 1, along with a number of accessories that can be included/useful in either the use and/or disposal of the panty/brief

### DETAILED DESCRIPTION OF THE PREFERRED EMBODIMENTS

The disposable, undergarment-like panty/brief of this invention can include by way of example, a number of features commonly found in disposable, personal care products commonly associated with a diaper for infants and/or adults. It is, however, understood that such similarities are superficial in that the undergarment-like, panty/brief of this invention is neither intended nor suitable for use as an alternative undergarment, nor is its contemplated for use in conjunction with an undergarment. The primary and intended purpose thereof is to an inexpensive and convenient substitute for a portable commode in a remote location where conventional toilet facilities are unavailable, (e.g. camping, hiking in the wilderness).

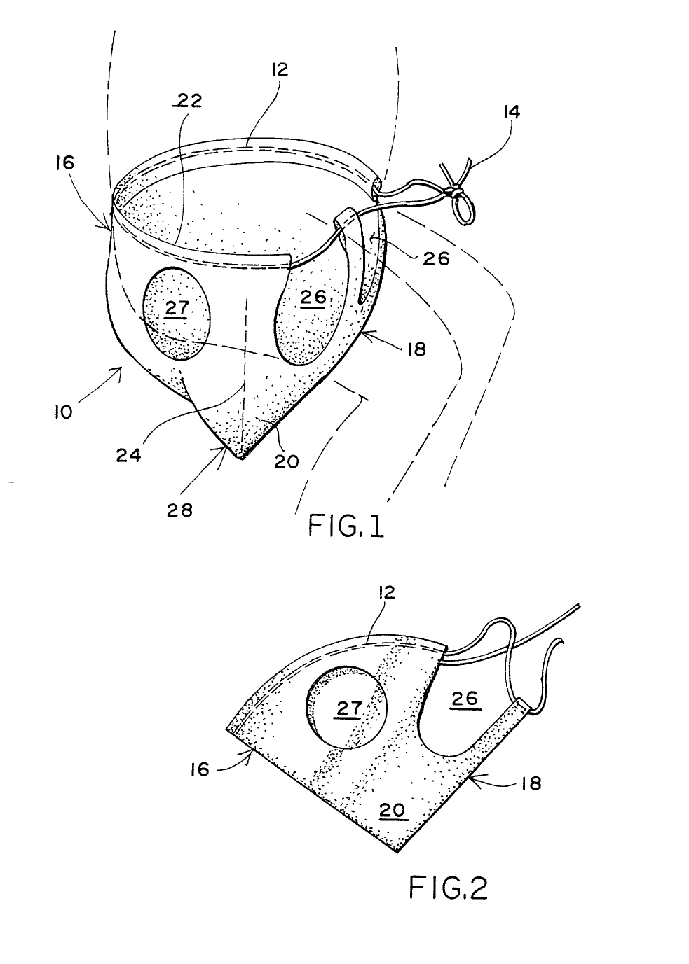

The basic configuration of the disposable, undergarment-like panty/brief of this invention are shown in FIG. 1. This panty/brief (10) includes a waist band (12) equipped with a draw-string (14), a back portion or panel (16), a front portion or panel (18), an extended seat/crotch/sump (20). Each of the back portion or panel (16) and front portion or panel (18) is attached at its corresponding top margins (22) to the waist band (12) and further cooperate with each other at each of its corresponding lateral margins (24), so as to generally define leg openings (26) in the undergarment-like panty/brief The front and back portions or panels are further attached to one another at each of its corresponding bottom margins (28) so as to define the extended crotch/seat/sump (20).

As illustrated in FIG. 2, the loosening of the draw string (14) also permits the release of a portion of the front panel (18) to allow for putting on and/or removal of the panty/brief without complete removal of other items of clothing. One of the primary design features of the panty/brief (10) illustrated in FIGS. 1 & 2 is the provision of an extended seat or crotch (20) to afford both the collection of human waste and thereby maintain it remote from the buttocks of the wearer. Upon completion of, for example, a bowel movement, the panty/brief permits the use of toilet tissue through an aperture (27) located in rear panel (16) of the panty/brief, and optionally located on the left hand side of the brief (10) (not shown). The toilet tissue is also discarded within the panty/brief. Upon completion of the use of the panty/brief, the drawstring (14) is loosened, the front panel (18) of the panty/brief thereby allowed to disengage the user's legs, removed from the user and sealed by twisting the waistband and tying the twisted waistband with the drawstring.

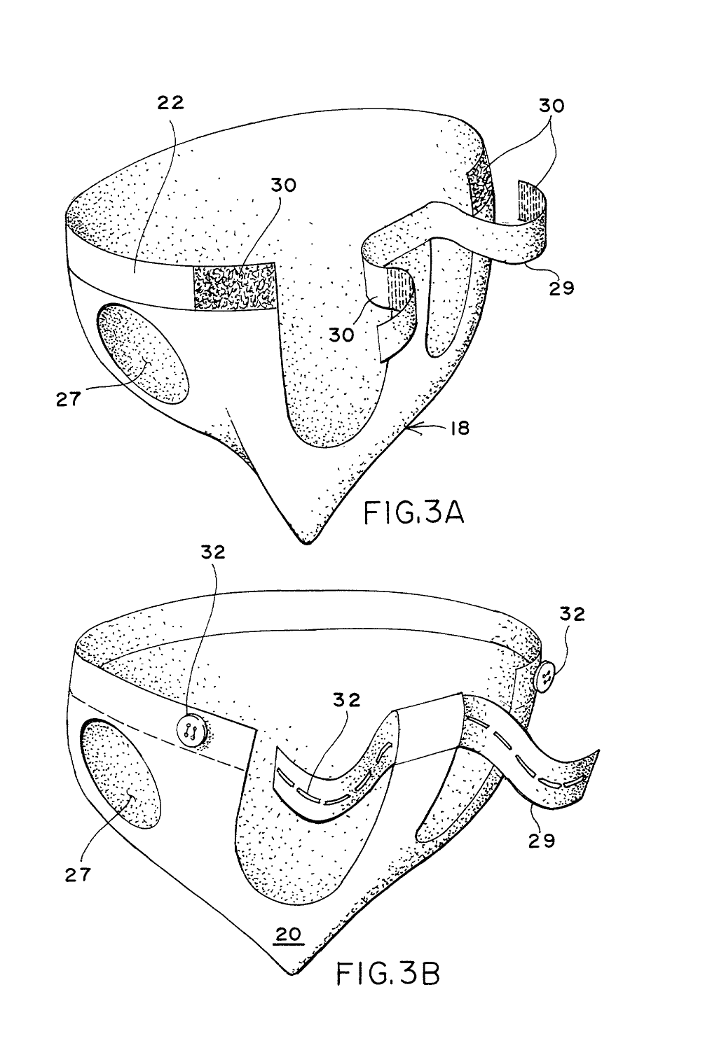

FIGS. 3A & 3B depict alternative embodiments of the panty/brief of FIG. 1 wherein the waistband (12) comprises a detachable belt (29) having a hook/loop fastener (30) (e.g. Velcro™) (FIG. 3A) or, a series of buttons/snaps (32) (FIG. 3B), designed to loosen and tightened the panty/brief about the waist of the user.

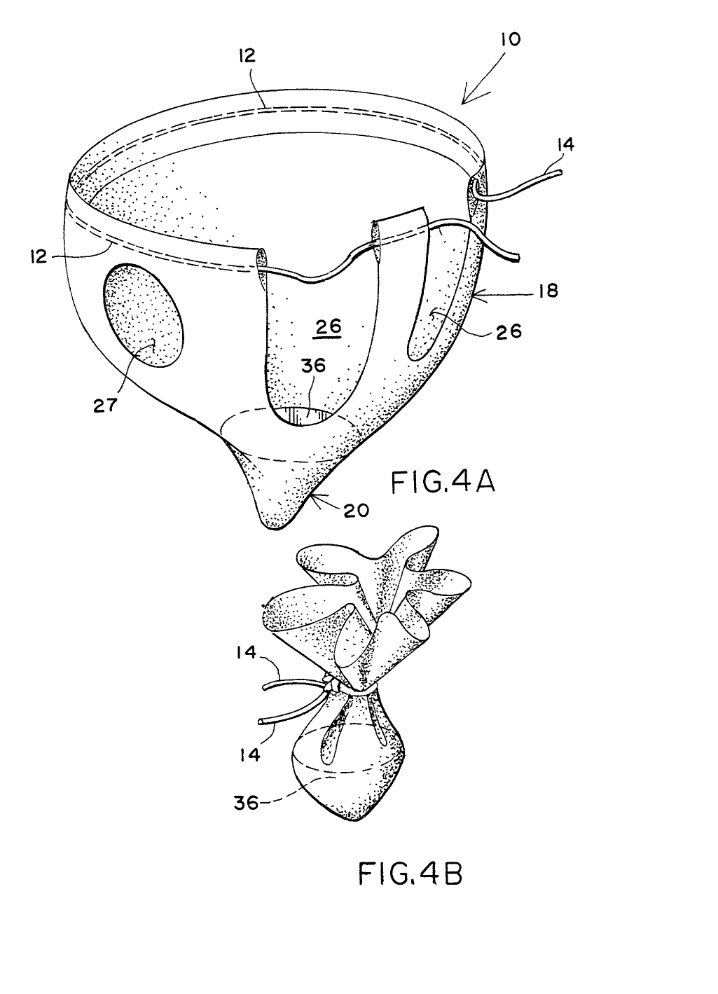

FIGS. 4A & 4B depict the panty/brief subsequent to use and upon removal by the user. The waste (36) (shown in phantom lines) remains in the extended seat or crotch (20) (FIG. 4A) and is thereafter sealed within the panty/brief by first twisting of the panty/brief (FIG. 4B) to confine the waste (36) in the extended seat/crotch (20) followed thereafter by tying the draw string (14) around the waist band (12) to insure sealing.

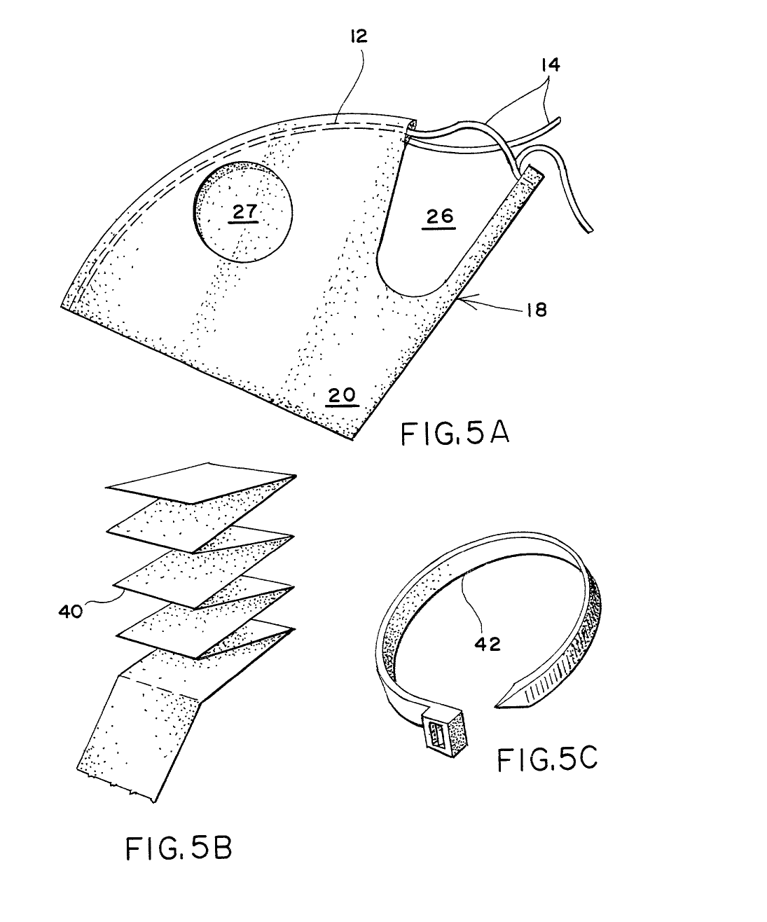

FIGS. 5A, 5B, and 5C depict a number of the accessories that can be pre-packaged with the panty/brief of this invention, including toilet tissue (40), closure straps (42) (similar to “railroad ties”) and numerous personal hygiene items (not shown).

What is claimed is:

1. In a disposable, garment-like panty/brief for collection and containment of human waste, the improvement comprising:
a disposable, undergarment-like panty/brief comprising a waist band having means for tightening and loosening said waste band, a back portion or panel, a front portion or panel, and a sump area,
each of said back portion or panel and said front portion or panel being attached at its corresponding top margins to said waist band and further cooperate with each other within said panty/brief so that (1) each of its corresponding lateral margins is attached to one another to generally define leg openings in the undergarment-like brief or panty, and (2) each of its corresponding bottom margins is attached to one another to generally define an extended crotch or seat, said extended sump area being further characterized as forming a container within said garment, remote from the buttocks of the wearer, for the collection of human waste.

2. The improved ensemble of claim 1, wherein the said back portion or panel of said panty/brief includes an aperture sufficient in size to permit the effective use of toilet tissue by the user while said garment is in place on the said user.

3. The improved ensemble of claim 1, wherein said panty/brief has a waist band which includes loop and hook fasteners for tightening and loosening said waist band around the waist of said user.

4. The improved ensemble of claim 1, wherein said panty/brief has a composite waist band comprising a first section having a belt-like strap associated with said front panel of said panty/brief intermediate between each of said leg openings, said belt-like strap having a series button holes for adjustable buttoning to a pair of buttons which are affixed to said waste band on either side of each of said leg openings.
